(19) United States
(12) Patent Application Publication
(10) Pub. No.: US 2025/0120684 A1
(43) Pub. Date: Apr. 17, 2025

(54) MAGNETIC COUPLING FOR SURGICAL INSTRUMENTS

(71) Applicant: Medos International Sàrl, Le Locle (CH)

(72) Inventors: Eric Biester, Barrington, RI (US); Michael Sorrenti, Middleboro, MA (US); Kevin Yeamans, Providence, RI (US); Rafael Garcia de Oliveira, Raynham, MA (US)

(21) Appl. No.: 18/911,428

(22) Filed: Oct. 10, 2024

Related U.S. Application Data
(60) Provisional application No. 63/589,618, filed on Oct. 11, 2023.

(51) Int. Cl. A61B 17/00

(52) U.S. Cl. CPC A61B 17/00; A61B 2017/00477; A61B 2017/00876

(57) ABSTRACT

Modular surgical instruments or components that utilize a magnetic coupling to more securely attach to other surgical instruments and components are disclosed. The devices can be modular drivers for use with surgical instruments that utilize an actuation force, such as a spinal rod reducer. In one example, a surgical instrument driver can include a non-magnetic body having a proximal end and a distal end with a distal-facing recess configured to receive a portion of a surgical instrument. The recess can include a surface configured to contact the portion of the surgical instrument disposed in the recess to impart torque thereto. The driver can further include a magnet disposed within the body such that the magnet is configured to extend proximal to a proximal end of a surgical instrument disposed within the recess and radially outward beyond the proximal end of the surgical instrument disposed within the recess.

### CROSS-REFERENCE TO RELATED APPLICATIONS

This application claims the benefit of U.S. Provisional Application No. 63/589,618, filed on Oct. 11, 2023. The entire contents of this application are incorporated by reference herein.

### FIELD

This disclosure relates generally to surgical instruments and, more particularly, to magnetic couplings for use with surgical instruments, such as couplings for use with surgical instrument drivers in a variety of settings, including orthopedic surgery.

### BACKGROUND

Surgical instruments are utilized in a variety of types of surgical procedures. In many surgical procedures, there can be a need to selectively couple various instruments or components to perform a task. For example, in many orthopedic procedures, a driver can be selectively coupled to an implant or surgical instrument to impart forces thereto, such as torque, etc.

In orthopedic surgery or neurosurgery in particular, fixation systems can be used to maintain a desired spatial relationship between multiple bones or bone fragments. For example, in spinal surgery, a spinal fixation system can be implanted into a patient to align and/or fix a desired orientation of one or more vertebrae. A typical spinal fixation system can include bone anchors implanted in the vertebrae and longitudinal rods, tethers, or other elements that are secured to the bone anchors by setscrews or other closure mechanisms. Implanting the fixation system can involve multiple steps, e.g., rod reduction to approximate the rod and implanted bone anchors, derotation to adjust positioning of one or more vertebrae, and setscrew insertion to lock the rod to the implanted bone anchors, among others.

Rod reduction, derotation, and setscrew management can involve the use of several surgical instruments and various drivers or other components that can be selectively coupled with other instruments or components to impart forces thereto, such as a torque, etc. Given space constraints and close placement of multiple instruments along a patient's spine, for example, larger driver handles configured to be grasped by a user can be modular and selectively coupled to a particular implant or instrument to avoid interfering with other instrumentation when not in use.

This approach can have certain drawbacks, however. For example, some devices utilize a slip-fit driver that is disposed over a portion of an instrument to be manipulated. A slip fit coupling, however, provides minimal security between the driver and the instrument during typical use of the instrument because, for example, even small axially directed forces can separate the two components. To address this, certain instruments include a more secure connection, such as fixing the driver to the surgical instrument. This, however, eliminates modularity of the driver such that it cannot be used with different surgical instruments during a procedure. This approach can also cause the interference issues noted above when multiple such instruments are implanted close together, such as when using rod reducers coupled to implanted pedicle screws along a patient's spine. Still other approaches utilize active mechanical locking mechanisms to selectively couple a driver to an instrument, but these mechanisms add bulk, in both occupied volume and weight, and can require additional interaction from the user to engage/disengage the locking mechanism.

Accordingly, there is a need for improved devices and methods for securing a modular component, such as a driver, to a surgical instrument, such as a reducer.

### SUMMARY

Disclosed herein are modular surgical instruments or components that utilize a magnetic coupling to more securely attach to other surgical instruments and components. The devices disclosed herein can have particular utility, for example, with regard to modular drivers for use with surgical instruments that utilize an actuation force, such as a spinal rod reducer. In one example, a surgical instrument driver can include a non-magnetic body having a proximal end and a distal end with a distal-facing recess configured to receive a portion of a surgical instrument. The recess can include a surface configured to contact the portion of the surgical instrument disposed in the recess to impart torque thereto. The driver can further include a magnet disposed within the body such that the magnet is configured to extend proximal to a proximal end of a surgical instrument disposed within the recess and radially outward beyond the proximal end of the surgical instrument disposed within the recess.

Any of a variety of alternative or additional features can be included and are considered within the scope of the present disclosure. For example, in some embodiments, the magnet can be a cylindrical magnet and a central axis of the cylindrical magnet can be substantially perpendicular to the central longitudinal axis of the body.

In certain embodiments, the magnet can be magnetized such that a radially inward portion of the magnet relative to the central longitudinal axis of the body has a first polarity and a radially outward portion of the magnet relative to the central longitudinal axis of the body has a second polarity opposite the first polarity. In some embodiments, the magnet can be disposed such that a distal portion of the magnet relative to the central longitudinal axis of the body is distal to the proximal end of the surgical instrument disposed within the recess and a proximal portion of the magnet relative to the central longitudinal axis of the body is proximal to the proximal end of the surgical instrument disposed within the recess.

In some embodiments, the magnet can be magnetized such that a proximal portion of the magnet relative to the central longitudinal axis of the body has a first polarity and a distal portion of the magnet relative to the central longitudinal axis of the body has a second polarity opposite the first polarity. In certain embodiments, the magnet can be further configured such that the distal portion of the magnet relative to the central longitudinal axis of the body is proximal to the proximal end of the surgical instrument disposed within the recess.

In certain embodiments, the body can include a central lumen extending from the proximal end of the body to the recess. In some embodiments, the magnet can be disposed outside the central lumen.

In some embodiments, the magnet can further comprise a plurality of magnets. In certain embodiments, the plurality of magnets can further comprise three magnets. In some embodiments, the plurality of magnets can be disposed around a circumference of the body.

In certain embodiments, an outer surface of the body can include a feature to facilitate a user gripping the body.

In some embodiments, the surface of the recess can be substantially planar. In certain embodiments, the recess can be substantially hexagonal with a plurality of substantially planar surfaces forming a circumference thereof.

In another example, a surgical instrument driver can include a modular handle body with a recess at a distal end of the body. The recess can extend through a portion of a length of the body and configured to engage with a proximal end of a surgical instrument. The instrument driver can also include a plurality of cylindrical magnets fixed within the body and configured to produce a magnetic field that urges a portion of the surgical instrument into the opening.

As with the example noted above, any of a variety of alternative or additional features can be included and are considered within the scope of the present disclosure. For example, in some embodiments, each magnet can be fixed within a bore formed in a distal portion of the modular handle body to locate the magnet in radially spaced-apart relation to a central longitudinal axis of the modular handle body.

In certain embodiments, the recess can include a hex shaped portion at a distal end thereof. In some embodiments, the plurality of cylindrical magnets can be magnetized axially such that a first circular surface of each magnet contains a magnetic charge opposing the charge of an opposing second circular surface of each magnet. In certain embodiments, a portion of the plurality of cylindrical magnets can overlap the hex shaped portion of the recess along a central longitudinal axis of the modular handle body.

In some embodiments, the plurality of cylindrical magnets can be magnetized diametrically such that a first curved side of each magnet contains a magnetic charge opposing the charge of a second opposing curved side of each magnet. In certain embodiments, the plurality of cylindrical magnets can be disposed proximal to the hex shaped portion of the recess.

The modular devices disclosed herein can be configured to couple to a proximal end of a surgical instrument, where it can remain secured during use of the instrument by a magnetic force or removed by an axial pull in the proximal direction that overcomes the magnetic force. The magnetic force can be provided by magnets embedded in a distal portion of the handle. Axially magnetized magnets can be disposed in the driver overlapping a proximal portion of an inserted instrument, such that a resulting magnetic force draws the instrument into the driver and secures it until a greater axial force is provided to separate the driver from the instrument. Alternatively, diametrically magnetized magnets can be disposed proximal to a proximal portion of an inserted instrument to provide a similar effect.

One example method of use can include distally advancing a surgical instrument driver over a proximal portion of a surgical instrument such that the proximal portion of the surgical instrument enters a recess formed in a distal end of the surgical instrument driver. The method can further include maintaining a position of the surgical instrument driver relative to the surgical instrument using a magnetic field created by a magnet disposed within the surgical instrument driver such that the magnet extends proximal to the proximal end of the surgical instrument disposed within the recess and radially outward beyond the proximal end of the surgical instrument disposed within the recess. The method can further include rotating the surgical instrument driver to impart torque to the surgical instrument.

As with the instruments described above, the methods disclosed herein can include any of a variety of additional or alternative steps that are considered within the scope of the present disclosure. For example, in some embodiments, the method can further include proximally withdrawing the surgical instrument driver from the surgical instrument with sufficient force to overcome the magnet field and separate the surgical instrument driver from the surgical instrument.

In certain embodiments, the method can further include advancing an instrument through a central lumen of the surgical instrument driver into a lumen of the surgical instrument.

In some embodiments, distally advancing the surgical instrument driver over a proximal portion of the surgical instrument can include contacting a ridge formed on the surgical instrument to a distal-facing surface of the surgical instrument driver.

In certain embodiments, distally advancing the surgical instrument driver over a proximal portion of the surgical instrument can include contacting a ridge formed at a proximal end of the recess to the proximal portion of the surgical instrument.

The devices and methods disclosed herein can provide a low profile, simplified interface with a secure connection to a surgical instrument, such as a spinal rod reducer, by utilizing magnets disposed in a distal portion of a polymer or other non-magnet body. In some embodiments, multiple cylindrical magnets that are magnetized through their thickness can be embedded into bores oriented symmetrically about a hex-shaped or other drive feature. The magnets can be axially disposed such that a portion of each magnet is proximal to a proximal-most portion of a surgical instrument that couples with the drive feature. This can maintain some level of resultant axial magnetic force between the driver and the surgical instrument, despite the magnetization being through the thickness of the magnets. Alternatively, magnets can be magnetized through their diameter in some embodiments and can be disposed entirely proximal to a proximal-most portion of a surgical instrument that couples with the drive feature.

Any of the features or variations described herein can be applied to any particular aspect or embodiment of the present disclosure in a number of different combinations. The absence of explicit recitation of any particular combination is due solely to avoiding unnecessary length or repetition.

### BRIEF DESCRIPTION OF THE DRAWINGS

The aspects and embodiments of the present disclosure can be more fully understood from the following detailed description taken in conjunction with the accompanying drawings, in which:

FIG. 1 is a perspective view of one embodiment of a modular surgical instrument driver coupled to a proximal end of one embodiment of a reducer instrument;

FIG. 2 is a proximal end view of the modular surgical instrument driver of FIG. 1;

FIG. 3 is a distal end view of the modular surgical instrument driver of FIG. 1;

FIG. 4 is a cross-sectional view of the modular surgical instrument driver of FIG. 1 taken along the line 4-4 in FIG. 1;

FIG. 5 is a detail view of a distal end surface of the modular surgical instrument driver of FIG. 1;

FIG. 6 is a perspective view of a portion of one embodiment of a reducer instrument;

FIG. 7 is a side view of the modular surgical instrument driver of FIG. 1;

FIG. 8 is a cross-sectional view of the modular surgical instrument driver of FIG. 1 taken along the line A-A in FIG. 7;

FIG. 9 is a cross-sectional view of the modular surgical instrument driver of FIG. 1 taken along the line 9-9 in FIG. 7;

FIG. 10 is a distal end perspective view of the modular surgical instrument driver of FIG. 1;

FIG. 11 is a side view of one embodiment of a modular surgical instrument driver;

FIG. 12 is a cross-sectional view of the modular surgical instrument driver of FIG. 11 taken along the line A-A in FIG. 11;

FIG. 13 is a cross-sectional view of the modular surgical instrument driver of FIG. 11 taken along the line 13-13 in FIG. 11;

FIG. 14 is a distal end view of the modular surgical instrument driver of FIG. 11;

FIG. 15 is a distal end perspective view of the modular surgical instrument driver of FIG. 11; and

FIG. 16 is a side view of the modular surgical instrument driver of FIG. 1 interfacing with the portion of the reducer instrument of FIG. 6.

### DETAILED DESCRIPTION

Certain example embodiments will now be described to provide an overall understanding of the principles of the structure, function, manufacture, and use of the devices, systems, and methods disclosed herein. One or more examples of these embodiments are illustrated in the accompanying drawings. The devices, systems, and methods specifically described herein and illustrated in the accompanying drawings are non-limiting embodiments. The features illustrated or described in connection with one embodiment may be combined with the features of other embodiments. Such modifications and variations are intended to be included within the scope of the present disclosure.

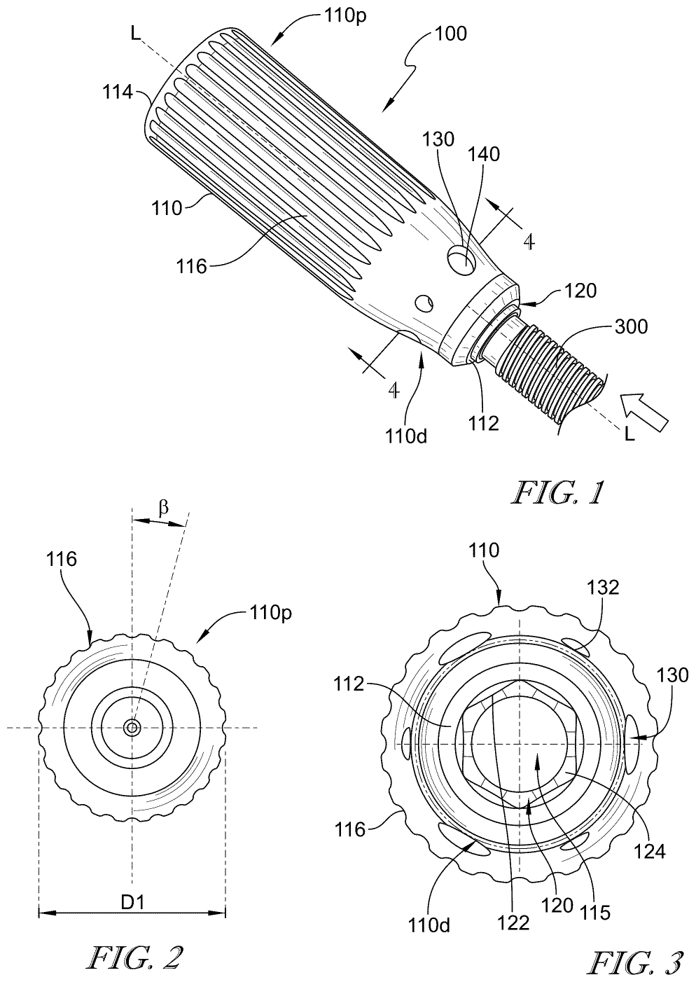

FIG. 1 illustrates a perspective view of one embodiment of a modular surgical instrument driver 100 coupled to the proximal end of a reducer instrument 300. While the illustrated instrument 300 is a reducer that can be utilized in a spinal surgery to urge a spinal fixation rod or other element into a recess of an implanted bone anchor, any of a variety of other instruments can be utilized in connection with the driver 100. Further, while one embodiment of the driver 100 is illustrated in FIG. 1, the magnetic couplings disclosed herein can be integrated into any of a variety of drivers or other surgical instruments that can have different form factors. This can include everything from T-shaped driver handles in favor of the elongated driver handle shown in FIG. 1, to ratcheting handles, powered drivers, etc.

The driver 100 can include a generally cylindrical body 110 with a tapered distal portion 110d. The body 110 can be made from a non-magnetic material, such as a polymer, etc., and can include one or more bores 130 formed in a distal portion thereof with one or more magnets 130 disposed therein. The distal end surface 112 of the body 110 can include a recess 120 in which at least a portion of the reducer 300 is received. The recess 120 can extend partially through a length of the body 110 along a longitudinal axis L thereof. In some embodiments, the recess can be in communication with a lumen 115 that extends to the proximal end 114 of the body 110.

An outer surface of the body 110 can include one or more surface features to facilitate gripping by a user. For example, the outer surface can be textured, faceted, knurled, grooved, etc. In the illustrated embodiment, a plurality of grooves 116 are formed around the outer circumference of the body. FIG. 2 illustrates the proximal end 114 of the body 110 and an end view of the grooves 116. In particular, there are 24 radially spaced grooves 116 formed around the circumference of the body 110 with centers separated from one another by an angle β of about 15 degrees, though any number of grooves or other features can be utilized, can include different spacing, etc. As shown in FIG. 1, the grooves 116 can begin at the proximal end 114 of the body 100 and terminate proximal to the tapered distal portion 110d. The proximal end 114 of the cylindrical body can be a generally planar surface and the body can have a maximum outer diameter D1 of about 30 mm, although a variety of differently sized bodies can be utilized based on a number of factors, such as user preference, size of instrument being utilized with the driver, etc.

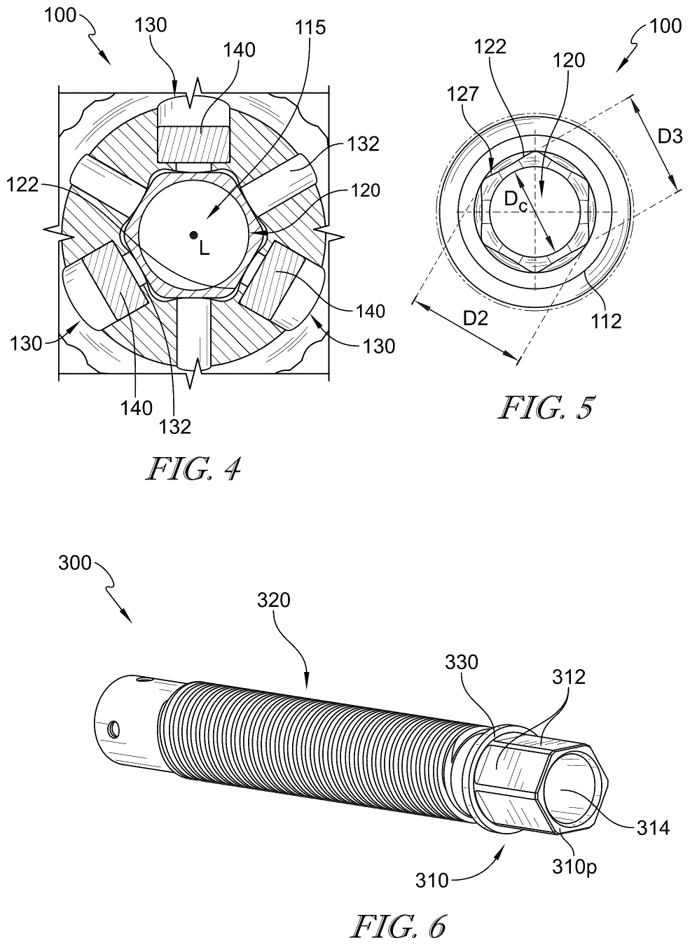

FIG. 3 illustrates a distal end view of the driver 100, FIG. 4 illustrates a cross sectional view of the driver 100 taken along the line 4-4 in FIG. 1, and FIG. 5 illustrates a detail view of the distal end surface 112 with recess 120. The recess 120 can include a surface 122 configured to contact a portion of a surgical instrument disposed in the recess to impart a force thereto, such as a torque, etc. In the illustrated embodiment, the inner surface of the recess 120 can include a plurality of substantially planar surfaces 122 arranged in a hexagonal shape and configured to contact a hexagonal drive feature 310 (see FIG. 6) formed on a proximal end of the reducer 300. In other embodiments, differently shaped drive features can be utilized. The hexagonal shape of the recess 120 and the drive feature 310 can prevent relative rotational movement therebetween. As shown in FIG. 5, the hexagonal recess 120 can be about 13 mm in diameter D2 between opposing corners 127, while the distance D3 between opposed planar surfaces 122 can be about 12 mm. As noted above, the noted dimensions are examples only and devices having a variety of different dimensions are also possible and within the scope of the present disclosure.

A proximal portion of one embodiment of a reducer instrument 300 is illustrated in FIG. 6. In particular, the illustrated instrument 300 can be a proximal portion of an inner reducer sleeve that can couple with a distal translating portion (not shown) and be threadably received within another sleeve (not shown) that couples to an implant, such as a bone anchor, etc. One example reducer instrument that includes a component like the instrument 300 of FIG. 6 is disclosed in U.S. Pat. Pub. No. 2022/0280207, entitled “Sequential Reducer,” the entire contents of which are incorporated by reference herein. A proximal portion of the instrument 300 can include a drive feature 310. In the illustrated embodiment, the drive feature 310 is a hexagonal protrusion having six substantially planar surfaces 312 disposed around the circumference thereof. The hexagonal drive feature 310 can be configured to be received within the recess 120 of the driver 100 such that the driver can be utilized to rotate the instrument 300. As noted above, in other embodiments different complementary shapes having one or more substantially planar surfaces configured to abut one another can be utilized. The proximal most surface 310p of the instrument 300 can include an opening into a lumen 314 extending through the instrument 300. A portion 320 of the instrument distal to the drive feature 310 can be threaded and a ridge 330 can be disposed between the drive feature 310 and threaded portions 320. The ridge 330 is configured to abut the distal end surface 112 of the body 110 to limit insertion of the drive feature 310 into the recess 120.

Returning to FIG. 4, magnets 140 can be positioned within the body 110 such that a resulting axial magnetic force attracts a proximal portion of a reducer instrument 300 into the recess 120. The magnets 140 can be disposed in bores 130 formed within the tapered distal portion 110d of the body 110. The bores 130 can extend from a surface of the body 110 towards the recess 120 and/or lumen 115, and can terminate a distance from the recess 120. In cases where multiple bores 130 receive multiple magnets 140, a distance between a distal or radially inward end of each bore 130 and the recess 120 can be consistent, ensuring placement of the magnets at equidistant positions relative to the central longitudinal axis L. This, along with placement at equally spaced intervals around a circumference of the driver 100 can create a balanced magnetic force within the recess 120. In some embodiments, a second bore 132 can extend from a distal or radially inward end of the bore 130 through to an outer surface of the body opposite the proximal or radially outward end of the bore 130. The second bore 132 can have a diameter smaller than the first bore 130 and magnet 140 and can provide a path for urging the magnet 140 proximally or radially outward within the bore 130 using, for example, a rod passed through the second bore 132 and into the first bore 130. In some embodiments, the second bores 132 can be omitted entirely, as there may be no need to remove the magnets once positioned within the body 110. Indeed, in certain embodiments it can be desirable to eliminate the bores 132 such that there are not pathways from the recess 120 or lumen 115 into the bores 130, which can require cleaning, complicate sterilization, etc.

The magnets 140 can be cylindrical in certain embodiments, though other shapes can be utilized as well. The same is true for the bores 130 into which the magnets are placed. Any of a variety of magnetic materials can be utilized for the magnets 140. For example, in some embodiments the magnets 140 can be neodymium magnets. The magnets can be permanent magnets, electromagnets, or any other material that produces a magnetic field.

The magnets 140 can be retained within the bores 130 using a variety of methods. For example, the bores 130 can be sized such that the magnets 140 are press-fit into place and do not readily move once positioned. In some embodiments, the bores 130 can be sealed after positioning the magnets 140 therein, e.g., using a pressed plug, a threaded fastener, or a filler material, such as epoxy, etc. Still further, in some embodiments the magnets 140 can be positioned in a mold and the driver body 110 can be over molded using a flowable polymer material, etc.

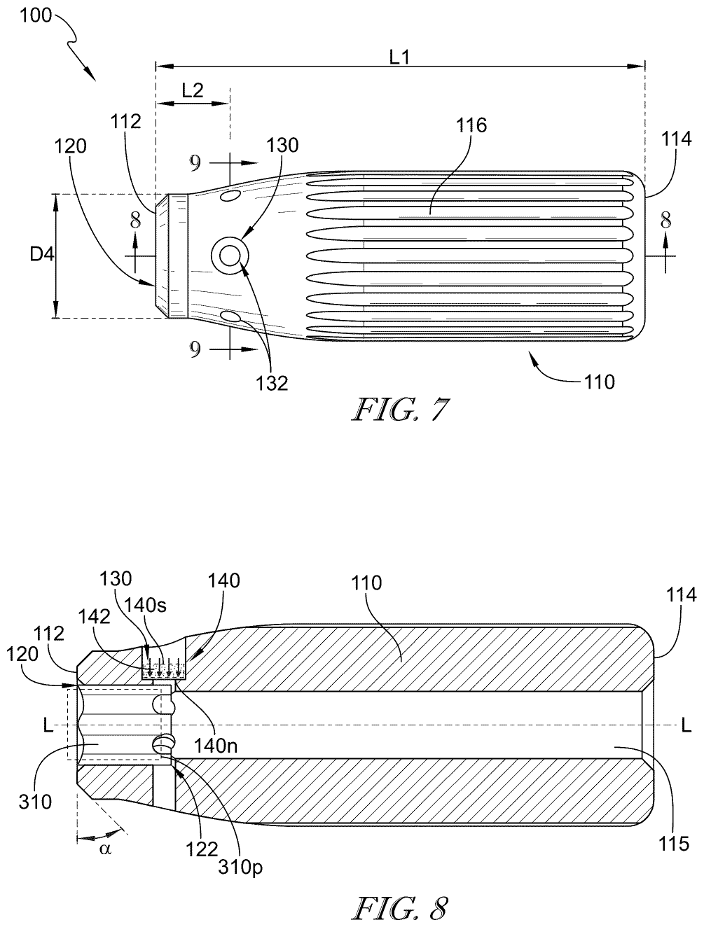

As shown in FIGS. 7 and 8, in some embodiments the cylindrical magnets 140 within the body 110 can be magnetized axially. As illustrated by arrows 142, one circular surface 140s of a magnet 140 can carry a first magnetic charge (e.g., positive or negative) while the opposing circular surface 140n can carry a second, opposite magnetic charge. In embodiments with multiple magnets disposed around a circumference of the body 110, all of the magnets can be disposed similarly, such that poles having a same charge can be positioned at a radially inward or radially outward position relative to the central longitudinal axis L.

Each magnet 140 can be disposed in the driver 100 such that a portion of the magnet 140 overlaps or lies in close proximity to a proximal end 310p of the reducer instrument 300 when the drive feature 310 is fully inserted into the recess 120. Said another way, the magnet 140 can be configured to extend proximal to the proximal end 310p of the surgical instrument 300 disposed within the recess 120. In this position, the magnets 140 can create a resultant magnetic force in a proximal direction along the central longitudinal axis L to urge the reducer 300 into the recess 120 of the drive 100. The magnetic force can also be sufficient to maintain the reducer 300 within the recess 120 during operation of the instrument 300, such that a clearly deliberate proximal force from a user is required to separate the driver 100 from the instrument 300.

FIG. 7 shows the outer surface of the generally cylindrical body 110 as described with respect to FIG. 1. The bores 130 can be formed in the distal end portion 110d of the body 110. The body length L1 can be about 85 mm in some embodiments, though other dimensions are possible and within the scope of the present disclosure. The bores 130 can be disposed at a length L2 from the distal end 112 of the body 110, which can be about 13 mm, though other dimensions are possible and within the scope of the present disclosure. The diameter D4 at the distal end 112 of the body 110 can be about 21 mm, though other dimensions are possible and within the scope of the present disclosure. FIG. 8 shows a cross-sectional view of the driver 100 taken along the line A-A of FIG. 7. As shown in FIG. 8, the body 110 can be tubular with the recess 120 extending therethrough along the longitudinal axis L and communicating with an inner lumen 115 extending from a proximal end of the body 110. The distal end 112 can include a beveled shoulder at an angle α of approximately 45 degrees, though other angles are possible and within the scope of the present disclosure.

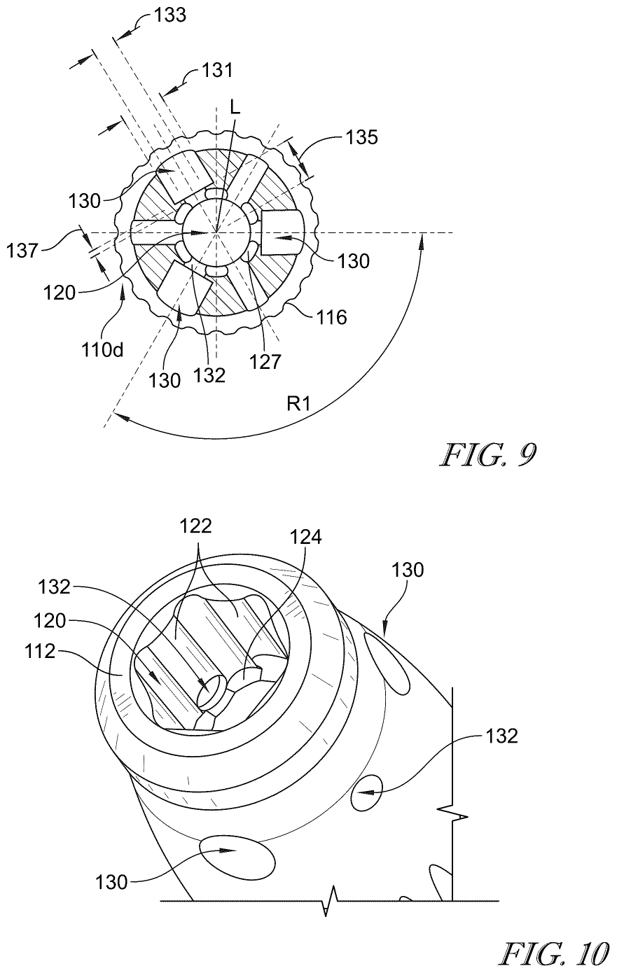

FIG. 9 illustrates a sectional view of the driver 100 taken along the line 9-9 of FIG. 7. As shown, three bores 130 can be disposed radially around the body 110d. The bores 130 can be evenly spaced from one another to create an even magnetic field through the recess 120. In the present embodiment, three bores 130 can be radially spaced at an angle R1 of about 120 degrees from one another. As previously described, additional bores 132 can be formed extending from the bores 130 to opposing sides of the body 110. The bores 132 can have a diameter 133 smaller than a diameter 131 of the bores 130 and smaller than a diameter of the magnets 140, such that the magnets 140 do not pass distally or radially inward beyond a distal or radially inward end of the bores 130. This can maintain each magnet 140 at a distance 135 from the central longitudinal axis L of the driver 100, with a wall thickness 137 separating the distal or radially inward end of the bore 130 from the recess 120. In some embodiments, the diameter 133 of the bores 132 can be about 3 mm while the diameter 131 of the bores 130 can be about 6 mm, though other dimensions can be utilized. In some embodiments, the distance 135 can be about 7 mm and the wall thickness 137 can be about 1 mm, though other dimensions can be utilized and are within the scope of the present disclosure. As shown in FIG. 4, the bores 130 can be formed such that they are aligned with the planar surfaces 122 of the recess 120.

FIG. 10 illustrates a front perspective view of the driver 100 and shows the bores 132 from within the recess 120 of the body 110. The recess 120 extends from the distal end surface 112 of the body 110 through a length of the distal portion 110d, though the recess can be made with different depths in other embodiments. The recess 120 includes a proximal end surface or ridge 124 that can serve as a step transition to the narrower lumen 115 extending farther proximally into the body 110. The ridge 124 can also serve as a maximum insertion stop for an instrument disposed in the recess, e.g., it can be configured to contact the proximal surface 310p of the reducer 300 to prevent further movement of the reducer in the proximal direction. Additionally or alternatively, maximum insertion can be achieved when the distal end surface 112 contacts a feature on the reducer 300, such as the ridge 330, as described above. In the illustrated embodiment, where axially magnetized magnets 140 are utilized, a portion of the bores 132 (and thereby the larger diameter bores 130 and the magnets 140 that are disposed in the bores 130) overlap both the recess and the proximal ridge 124 of the recess, as shown in the figure.

FIGS. 11-15 illustrate an alternative embodiment of a driver 200 that utilizes magnets 240 that are magnetized in a different manner from the magnets 140 of the driver 100. The driver 200 can have a similar shape, size, and features as the driver 100 described above. For example, the driver 200 can include a body 210 with a lumen 215 extending there through and a distal-facing recess 220 formed in a distal end surface 212. The body 210 can also include bores 230 formed therein that are configured to receive magnets 240 and smaller bores 232 extending from a distal end of the bores 230 to an opposing outer surface of the body 210. As a result, a detailed description of the structure and function of the driver 200 is omitted here for the sake of brevity and to focus on distinctions relative to the above-described embodiments.

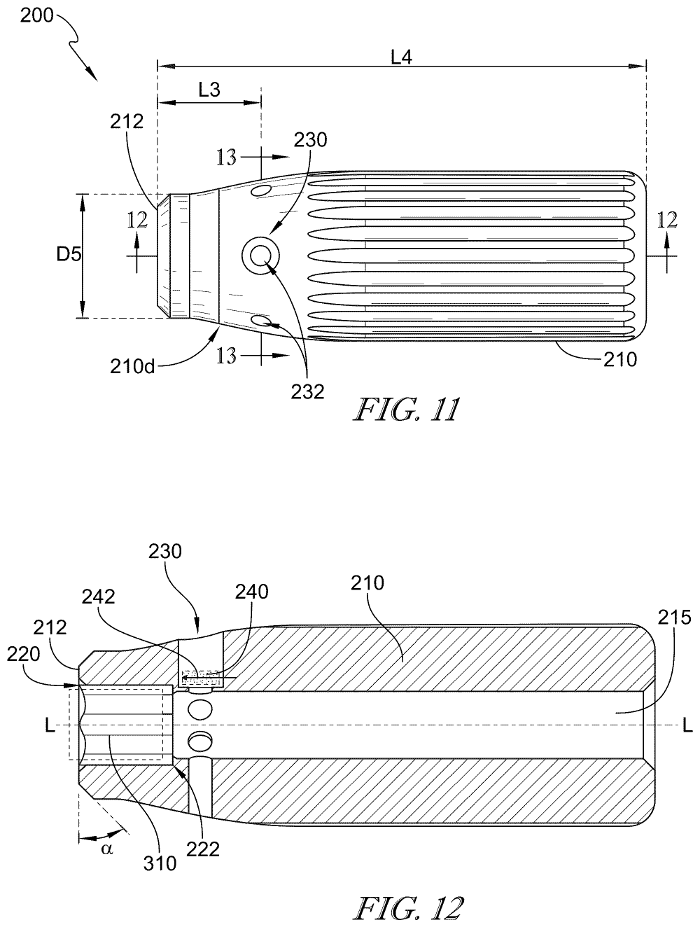

One such distinction relates to the one or more magnets 240 disposed within the body 210, which can be cylindrical as with the driver 100, but can be magnetized diametrically, such that one curved end carries a first magnetic charge (e.g., positive or negative) while the opposing curved end carries an opposite magnetic charge. This is illustrated by arrow 242 in FIG. 12, where FIG. 12 shows a cross-sectional view of the driver 200 taken along the line A-A in FIG. 11. In other words, the magnets 240 can be magnetized such that a proximal portion of the magnet relative to a central longitudinal axis L of the body 210 has a first polarity (e.g., positive or negative) and a distal portion of the magnet relative to the central longitudinal axis L of the body 210 has a second polarity opposite the first polarity.

As shown in FIG. 12, the magnets 240 can be positioned entirely behind the termination of the drive feature 310 or proximal end of the steel reducer 300 when it is disposed in the recess 220 of the driver 200. The more proximal positioning of the magnets 240 relative to the magnets 140 in the driver 100 described above can be required due to the manner of magnetization in order to provide a desired resultant magnetic force in a proximal direction along the longitudinal axis L of the driver 200, thereby drawing a surgical instrument (e.g., the reducer 300) into the recess 220. As shown in FIGS. 11 and 12, first bores 230 can be formed in the body 210 along a distal end portion 210d at a length L3 from a distal end surface 212 of the body 210 that has an overall length L4 and a diameter D5 at the distal end 212 of the body 210. In some embodiments, the distance L3 can be about 18 mm and the distance L4 can be about 85 mm, though other dimensions can be utilized and are within the scope of the present disclosure. The bores 230 can be disposed such that a magnet 240 disposed therein is positioned such that a distal portion of the magnet relative to the central longitudinal axis L of the body 210 is proximal to the proximal end of a portion 310 of a surgical instrument disposed within the recess. In other words, the magnet 240 does not overlap any portion of the surgical instrument disposed within the recess. In some embodiments, and as illustrated in FIG. 12, the magnet 240 does not overlap any portion of the recess 220 and is formed entirely proximal thereto. The bores 230 can be otherwise structured as described with respect to those of the driver 100, e.g., to seat the magnets 240 within the handle body 210 such that they are evenly spaced from the recess 220 to create a balanced magnetic field within the recess 220.

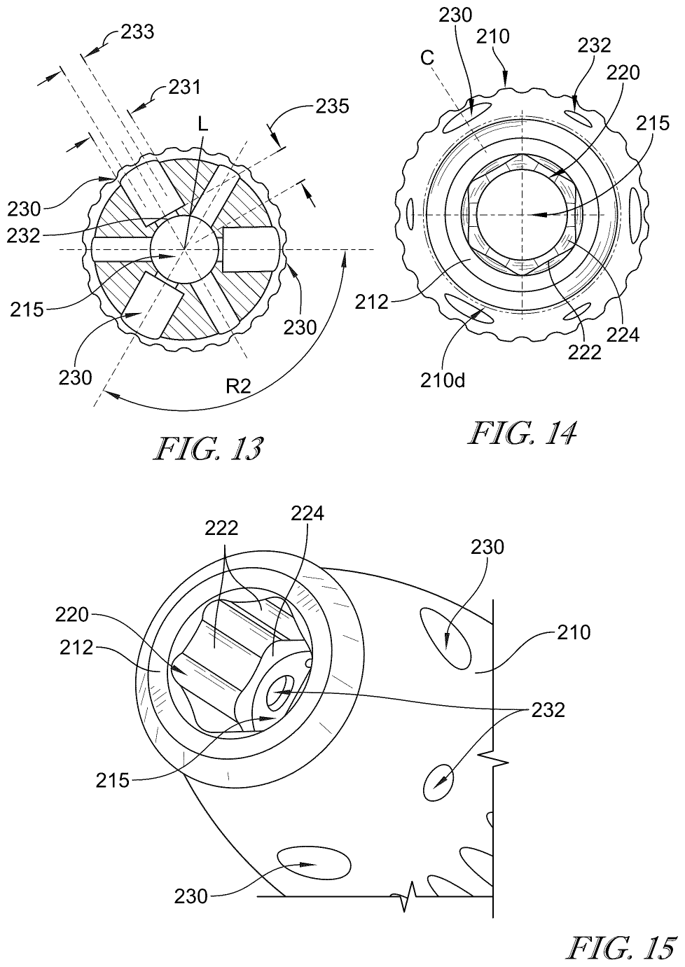

FIG. 13 shows a cross sectional view of the driver 200 taken along the line 13-13 of FIG. 11. As shown, the bores 230 can be positioned around the inner lumen 215 that extends proximal to the recess 220. In the present embodiment, three bores 230 can be radially spaced at an angle R2 of about 120 degrees from one another around the circumference of the driver 200. Additional bores 232 can extend from the bores 230 to opposing sides of the body 210. The bores 232 can have a diameter 233 smaller than a diameter 231 of the bores 230 and smaller than a diameter of the magnets 240, such that the magnets 240 do not pass distally or radially inward beyond a distal or radially inward end of the bores 230. This can maintain each magnet 240 at a distance 235 from the central longitudinal axis L of the driver 200. In some embodiments, the distance 235 can be about 6 mm, the diameter 233 can be about 3 mm, and the diameter 231 can be about 6 mm, though other dimensions can be utilized and are within the scope of the present disclosure.

FIG. 14 shows a distal end view of the driver 200, including the recess 220 extending into the distal end surface 212. The bores 230 are shown formed in an outer surface of the body 210 and aligned with substantially planar surfaces 222 of the hex-shaped recess 220 along the axis C. The bores 230 seat the magnets 240 within the body 210 at a position that provides sufficient magnetic force to urge the hex drive feature 310 of the reducer 300 or other surgical instrument into the recess 220 and maintain it in position during use until a user imparts sufficient axial force to overcome the magnetic force, thereby separating the driver 200 from the reducer 300.

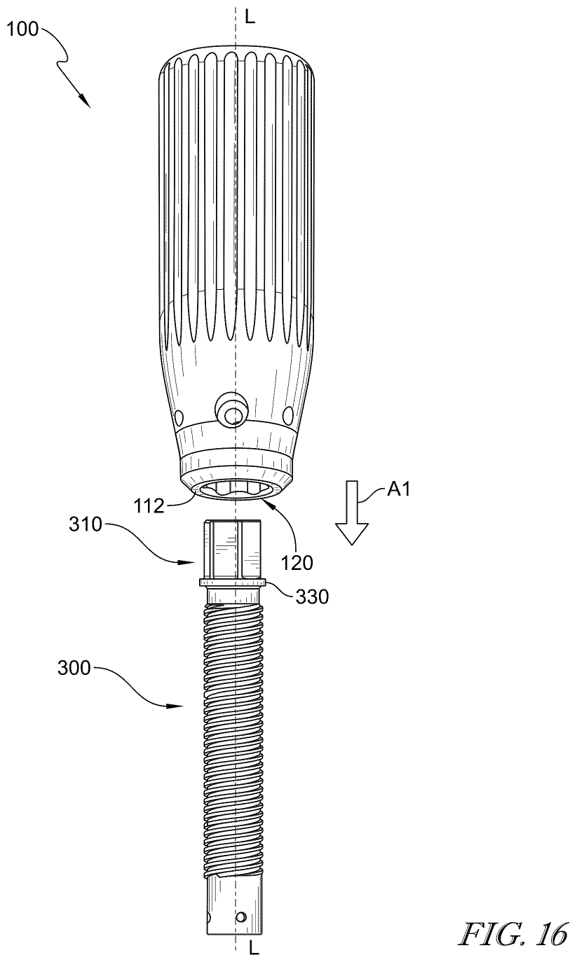

FIG. 15 illustrates a front perspective view of the driver 200 and shows the bores 232 from within the recess 220 of the body 210. The recess 220 extends from the distal end surface 212 of the body 210 through a length of the distal end portion 210d, though the recess can be made with different depths in other embodiments. The bores 230, along with the additional bores 232 extending therefrom that are visible within the lumen 215, can be positioned entirely proximal to the ridge 224 that defines the proximal end of the recess 220 and the transition to the lumen 215. In some embodiments, however, the recess 220 can have a different depth and may extend beyond the position of the magnets 240 and bores 230, 232. Regardless, the magnets 240 and bores 230, 232 can be positioned such that they remain entirely proximal to a proximal end of a portion of a surgical instrument disposed within the recess 220.

During use of the instruments disclosed herein, a user can couple the driver 100 to a surgical instrument, e.g., the reducer 300, by inserting the proximal drive feature 310 of the reducer into the distal-facing recess 120 of the driver 100, as shown in FIG. 16. This can be done, for example, by distally advancing the driver 100 in the direction of arrow Al along the longitudinal axis L over a proximal portion 310 of the surgical instrument 300 such that the proximal portion 310 of the surgical instrument 300 enters the recess 120. As the two components become closer, the instrument 300 can enter the magnetic field created by the magnets 140. The resultant magnetic force from the fields created by the magnets 140 can urge the instrument proximally into the recess 120 until the instrument is fully seated within the recess. This can occur, for example, when a proximal end of the surgical instrument contacts a proximal end of the recess (e.g., ridge 124) or when the distal end surface 112 contacts a feature on the surgical instrument (e.g., ridge 330). The magnetic force can also provide tactile feedback to a user regarding the coupling of the driver 100 and reducer 300, as the components can appear to “snap” into place when brought close together. The resultant magnetic force can also maintain the reducer 300 relative to the driver 100 and prevent inadvertent axial separation between the two components during use, while the one or more flat surfaces of the driver recess 120 and reducer drive feature 310 (e.g., hex-shaped flats in the illustrated embodiment) can prevent rotational movement between the components and allow the driver 100 to be grasped and utilized to rotate the reducer 300.

In some cases, it may be desirable to introduce one or more additional instruments through the lumen 115 of the driver 100 and into the lumen 314 of the reducer 300. The lumen 115 extending through the driver 100 that is in communication with the recess 120 can provide such functionality. Alternatively, the driver 100 can be separated or decoupled from the reducer 300 to allow such access to the lumen 314 of the reducer 300. The modular nature of the driver 100 can allow multiple repeated couplings and decouplings with one or more surgical instruments throughout the course of a procedure.

When separation of the driver 100 from the reducer 300 is desired, a user can apply a proximally directed axial force to the driver 100 that is of sufficient strength to overcome the resultant magnetic force maintaining the positioning of the components relative to one another. This can result in separation of the driver 100 from the reducer 300.

Various devices and methods disclosed herein can be used in minimally invasive surgery and/or open surgery. While various devices and methods disclosed herein are generally described in the context of surgery on a human patient, the methods and devices disclosed herein can be used in any of a variety of surgical procedures with any human or animal subject, or in non-surgical procedures.

Various devices disclosed herein can be constructed from any of a variety of known materials. Example materials include those that are suitable for use in surgical applications, including metals such as stainless steel, titanium, titanium nitride, nickel, cobalt, chrome, cobalt-chromium, or alloys and combinations thereof, polymers such as PEEK, ceramics, carbon fiber, and so forth. The various components of the devices disclosed herein can be rigid or flexible. In addition, one or more of the components or devices disclosed herein can be formed as monolithic or unitary structures, e.g., formed from a single continuous material, or can be formed from separate components coupled together in a variety of manners that either facilitate or discourage subsequent separation. One or more components or portions of the device can be formed from a radiopaque material to facilitate visualization under fluoroscopy and other imaging techniques, or from a radiolucent material so as not to interfere with visualization of other structures. Example radiolucent materials include carbon fiber and high-strength polymers. Further, various methods of manufacturing can be utilized, including 3D printing or other additive manufacturing techniques, as well as more conventional manufacturing techniques, including molding, stamping, casting, machining, etc.

Various devices or components disclosed herein can be designed to be disposed of after a single use, or they can be designed to be used multiple times. In either case, however, various devices or components can be reconditioned for reuse after at least one use. Reconditioning can include any combination of the steps of disassembly, followed by cleaning or replacement of particular pieces, and subsequent reassembly. In particular, a device or component can be disassembled, and any number of the particular pieces or parts thereof can be selectively replaced or removed in any combination. Upon cleaning and/or replacement of particular parts, the device or component can be reassembled for subsequent use either at a reconditioning facility, or by a surgical team immediately prior to a surgical procedure. Reconditioning of a device or component can utilize a variety of techniques for disassembly, cleaning/replacement, and reassembly. Use of such techniques, and the resulting reconditioned device or component, are within the scope of the present disclosure.

Various devices or components described herein can be processed before use in a surgical procedure. For example, a new or used device or component can be obtained and, if necessary, cleaned. The device or component can be sterilized. In one sterilization technique, the device or component can be placed in a closed and sealed container, such as a plastic or TYVEK bag. The container and its contents can be placed in a field of radiation that can penetrate the container, such as gamma radiation, x-rays, or high-energy electrons. The radiation can kill bacteria on the device or component and in the container. The sterilized device or component can be stored in the sterile container. The sealed container can keep the device or component sterile until it is opened in the medical facility. Other forms of sterilization are also possible, including beta or other forms of radiation, ethylene oxide, steam, or a liquid bath (e.g., cold soak). Certain forms of sterilization may be better suited to use with different devices or components, or portions thereof, due to the materials utilized, the presence of electrical components, etc.

In this disclosure, articles “a” and “an” are used herein to refer to one or to more than one (i.e., at least one) of the grammatical object of the article. By way of example, “an element” means at least one element and can include more than one element. The term “about” is used to provide flexibility to a numerical range endpoint by providing that a given value may be “slightly above” or “slightly below” the endpoint without affecting the desired result. The use herein of the terms “including,” “comprising,” or “having,” and variations thereof, is meant to encompass the elements listed thereafter and equivalents thereof, as well as additional elements. As used herein, “and/or” refers to and encompasses any and all possible combinations of one or more of the associated listed items, as well as the lack of combinations where interpreted in the alternative (“or”). Further, phrases such as “at least one of” or “one or more of”' may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B,” “one or more of A and B,” and “A and/or B” are each intended to mean “A alone, B alone, or A and B together.” A similar interpretation is also intended for lists including three or more items. For example, the phrases “at least one of A, B, and C,” “one or more of A, B, and C,” and “A, B, and/or C” are each intended to mean “A alone, B alone, C alone, A and B together, A and C together, B and C together, or A and B and C together.” In addition, use of the term “based on,” is intended to mean, “based at least in part on,” such that an un-recited feature or element is also permissible.

To the extent that linear, circular, or other dimensions are used in the description of the disclosed devices and methods, such dimensions are not intended to limit the types of shapes that can be used in conjunction with such devices and methods. Equivalents to such dimensions can be determined for different geometric shapes, etc. Further, like-numbered components of the embodiments can generally have similar features. Still further, sizes and shapes of the devices, and the components thereof, can depend at least on the anatomy of the subject in which the devices will be used, the size and shape of objects with which the devices will be used, and the methods and procedures in which the devices will be used.

The figures provided herein are not necessarily to scale. Still further, to the extent arrows are used to describe a direction of movement, these arrows are illustrative and in no way limit the direction that the respective component can or should be moved. Other movements and directions may be possible to create the desired result in view of the present disclosure. Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Further features and advantages based on the above-described embodiments are possible and within the scope of the present disclosure. Accordingly, the disclosure is not to be limited by what has been particularly shown and described. All publications and references cited herein are expressly incorporated herein by reference in their entirety, except for any definitions, subject matter disclaimers, or disavowals, and except to the extent that the incorporated material is inconsistent with the express disclosure herein, in which case the language in this disclosure controls.

Examples of the above-described embodiments can include the following:

1. A surgical instrument driver, comprising:
a non-magnetic body having a proximal end and a distal end with a distal-facing recess configured to receive a portion of a surgical instrument, the recess including a surface configured to contact the portion of the surgical instrument disposed in the recess to impart torque thereto; and
a magnet disposed within the body such that the magnet is configured to extend proximal to a proximal end of a surgical instrument disposed within the recess and radially outward beyond the proximal end of the surgical instrument disposed within the recess.

2. The device of example 1, wherein the magnet is a cylindrical magnet and a central axis of the cylindrical magnet is substantially perpendicular to the central longitudinal axis of the body.

3. The device of any of examples 1 to 2, wherein the magnet is magnetized such that a radially inward portion of the magnet relative to the central longitudinal axis of the body has a first polarity and a radially outward portion of the magnet relative to the central longitudinal axis of the body has a second polarity opposite the first polarity.

4. The device of example 3, wherein the magnet is disposed such that a distal portion of the magnet relative to the central longitudinal axis of the body is distal to the proximal end of the surgical instrument disposed within the recess and a proximal portion of the magnet relative to the central longitudinal axis of the body is proximal to the proximal end of the surgical instrument disposed within the recess.

5. The device of any of examples 1 to 2, wherein the magnet is magnetized such that a proximal portion of the magnet relative to the central longitudinal axis of the body has a first polarity and a distal portion of the magnet relative to the central longitudinal axis of the body has a second polarity opposite the first polarity.

6. The device of example 5, wherein the magnet is further configured such that the distal portion of the magnet relative to the central longitudinal axis of the body is proximal to the proximal end of the surgical instrument disposed within the recess.

7. The device of any of examples 1 to 6, wherein the body includes a central lumen extending from the proximal end of the body to the recess.

8. The device of example 7, wherein the magnet is disposed outside the central lumen.

9. The device of any of examples 1 to 8, wherein the magnet further comprises a plurality of magnets.

10. The device of example 9, wherein the plurality of magnets further comprises three magnets.

11. The device of any of examples 9 to 10, wherein the plurality of magnets are disposed around a circumference of the body.

12. The device of any of examples 1 to 11, wherein an outer surface of the body includes a feature to facilitate a user gripping the body.

13. The device of any of examples 1 to 12, wherein the surface of the recess is substantially planar.

14. The device of example 13, wherein the recess is substantially hexagonal with a plurality of substantially planar surfaces forming a circumference thereof.

15. A surgical instrument driver, comprising:
a modular handle body with a recess at a distal end of the body, the recess extending through a portion of a length of the body and configured to engage with a proximal end of a surgical instrument; and
a plurality of cylindrical magnets fixed within the body and configured to produce a magnetic field that urges a portion of the surgical instrument into the opening.

16. The device of example 15, wherein each magnet is fixed within a bore formed in a distal portion of the modular handle body to locate the magnet in radially spaced-apart relation to a central longitudinal axis of the modular handle body.

17. The device of any of examples 15 to 16, wherein the recess includes a hex shaped portion at a distal end thereof.

18. The device of example 17, wherein the plurality of cylindrical magnets are magnetized axially such that a first circular surface of each magnet contains a magnetic charge opposing the charge of an opposing second circular surface of each magnet.

19. The device of example 18, wherein a portion of the plurality of cylindrical magnets overlap the hex shaped portion of the recess along a central longitudinal axis of the modular handle body.

20. The device of example 17, wherein the plurality of cylindrical magnets are magnetized diametrically such that a first curved side of each magnet contains a magnetic charge opposing the charge of a second opposing curved side of each magnet.

21. The device of example 20, wherein the plurality of cylindrical magnets are disposed proximal to the hex shaped portion of the recess.

22. A surgical method, comprising:
distally advancing a surgical instrument driver over a proximal portion of a surgical instrument such that the proximal portion of the surgical instrument enters a recess formed in a distal end of the surgical instrument driver;
maintaining a position of the surgical instrument driver relative to the surgical instrument using a magnetic field created by a magnet disposed within the surgical instrument driver such that the magnet extends proximal to the proximal end of the surgical instrument disposed within the recess and radially outward beyond the proximal end of the surgical instrument disposed within the recess;
rotating the surgical instrument driver to impart torque to the surgical instrument.

23. The method of example 22, further comprising proximally withdrawing the surgical instrument driver from the surgical instrument with sufficient force to overcome the magnet field and separate the surgical instrument driver from the surgical instrument.

24 The method of any of examples 22 to 23, further comprising advancing an instrument through a central lumen of the surgical instrument driver into a lumen of the surgical instrument.

25 The method of any of examples 22 to 24, wherein distally advancing the surgical instrument driver over a proximal portion of the surgical instrument further comprises contacting a ridge formed on the surgical instrument to a distal-facing surface of the surgical instrument driver.

26. The method of any of examples 22 to 24, wherein distally advancing the surgical instrument driver over a proximal portion of the surgical instrument further comprises contacting a ridge formed at a proximal end of the recess to the proximal portion of the surgical instrument.

1. A surgical instrument driver, comprising:
a non-magnetic body having a proximal end and a distal end with a distal-facing recess configured to receive a portion of a surgical instrument, the recess including a surface configured to contact the portion of the surgical instrument disposed in the recess to impart torque thereto; and
a magnet disposed within the body such that the magnet is configured to extend proximal to a proximal end of a surgical instrument disposed within the recess and radially outward beyond the proximal end of the surgical instrument disposed within the recess.

2. The device of claim 1, wherein the magnet is a cylindrical magnet and a central axis of the cylindrical magnet is substantially perpendicular to a central longitudinal axis of the body.

3. The device of claim 1, wherein the magnet is magnetized such that a radially inward portion of the magnet relative to a central longitudinal axis of the body has a first polarity and a radially outward portion of the magnet relative to the central longitudinal axis of the body has a second polarity opposite the first polarity.

4. The device of claim 3, wherein the magnet is disposed such that a distal portion of the magnet relative to the central longitudinal axis of the body is distal to the proximal end of the surgical instrument disposed within the recess and a proximal portion of the magnet relative to the central longitudinal axis of the body is proximal to the proximal end of the surgical instrument disposed within the recess.

5. The device of claim 1, wherein the magnet is magnetized such that a proximal portion of the magnet relative to a central longitudinal axis of the body has a first polarity and a distal portion of the magnet relative to the central longitudinal axis of the body has a second polarity opposite the first polarity.

6. The device of claim 5, wherein the magnet is further configured such that the distal portion of the magnet relative to the central longitudinal axis of the body is proximal to the proximal end of the surgical instrument disposed within the recess.

7. The device of claim 1, wherein the body includes a central lumen extending from the proximal end of the body to the recess.

8. The device of claim 7, wherein the magnet is disposed outside the central lumen.

9. The device of claim 1, wherein the magnet further comprises a plurality of magnets.

10. The device of claim 9, wherein the plurality of magnets further comprises three magnets.

11. The device of claim 9, wherein the plurality of magnets are disposed around a circumference of the body.

12. The device of claim 1, wherein an outer surface of the body includes a feature to facilitate a user gripping the body.

13. The device of claim 1, wherein the surface of the recess is substantially planar.

14. The device of claim 13, wherein the recess is substantially hexagonal with a plurality of substantially planar surfaces forming a circumference thereof.

15. A surgical instrument driver, comprising:
a modular handle body with a recess at a distal end of the body, the recess extending through a portion of a length of the body and configured to engage with a proximal end of a surgical instrument; and
a plurality of cylindrical magnets fixed within the body and configured to produce a magnetic field that urges a portion of the surgical instrument into the opening.

16. The device of claim 15, wherein each magnet is fixed within a bore formed in a distal portion of the modular handle body to locate the magnet in radially spaced-apart relation to a central longitudinal axis of the modular handle body.

17. The device of claim 15, wherein the recess includes a hex shaped portion at a distal end thereof.

18. The device of claim 17, wherein the plurality of cylindrical magnets are magnetized axially such that a first circular surface of each magnet contains a magnetic charge opposing the charge of an opposing second circular surface of each magnet.

19. The device of claim 18, wherein a portion of the plurality of cylindrical magnets overlap the hex shaped portion of the recess along a central longitudinal axis of the modular handle body.

20. The device of claim 17, wherein the plurality of cylindrical magnets are magnetized diametrically such that a first curved side of each magnet contains a magnetic charge opposing the charge of a second opposing curved side of each magnet.

21. The device of claim 20, wherein the plurality of cylindrical magnets are disposed proximal to the hex shaped portion of the recess.

22.-26. (canceled)
